(19) United States
(12) Patent Application Publication
(10) Pub. No.: US 2020/0289309 A1
(43) Pub. Date: Sep. 17, 2020

(54) ORAL APPLIANCE

(71) Applicant: Mitsui Chemicals, Inc., Tokyo (JP)

(72) Inventors: Takashi YUKITA, Chiba-shi (JP); Yuki Iwabuchi, Chiba-shi (JP); Akira HASEGAWA, Chiba-shi (JP)

(21) Appl. No.: 16/354,224

(22) Filed: Mar. 15, 2019

(51) Int. Cl. A61F 5/56

(52) U.S. Cl. CPC A61F 5/566

(57) ABSTRACT

An oral appliance may include a first jaw retainer piece, a second jaw retainer piece, a holding member, and a rod. The holding member may be mounted on a front-facing portion of the first jaw retainer piece, and have an opening for receiving a rod. The rod may have a leading end slidably inserted into the opening of the holding member and a trailing end mounted on a front-facing portion of the second jaw retainer piece. The oral appliance may further include a restrictor that restricts sliding of the rod relative to the holding member.

### TECHNICAL FIELD

The present invention relates to an oral appliance for preventing and treating sleep apnea. More particularly, the present invention relates to an oral appliance for treatment of snoring and sleep apnea which will effectively maintain the mandible in a preferred anterior position, but which will also allow a limited degree of forward or lateral excursion of the mandible relative to the upper jaw.

### BACKGROUND

While people are asleep, the muscles relax causes the airways to close and vibrate as air is passed. This vibration causes the noise known as snoring. Also, the obstruction of the airways causes insufficient ventilation of the lungs, leading to disturbed sleep, and, potentially, to the person's breathing to stop for a moment (this being known as sleep apnea syndrome (SAS)).

SAS is a condition characterized by multiple episodes of cessation of respiration (apnea) during sleep, resulting from partial or complete obstruction of the airway by the root of the tongue and/or the soft palate falling down therein. As a result, sleep is disturbed and daytime sleepiness occurs. In recent years a large number of accidents have been attributable to this condition, including, for example, where the sufferer of the condition unknowingly drives a car while asleep.

The conventional method of treating SAS involves having the sufferer wear in his or her mouth while sleeping a mandibular advancement device, specifically an oral appliance, which applies pressure in such a manner to cause the lower jaw to be slightly drawn (pulled) forward from the normal occlusal position to forcibly maintain an airway, to prevent apnea during sleep.

More specifically, the oral appliance causes a slight forward movement of the mandible with respect to the upper maxillary bone, opening a bigger space at the back of the oral cavity, hence facilitating the passage of air from and to the pharynx. Thus, the mandibular advancement device can prevent the constriction of the airway, a breathing stop or low respiration during sleep.

However, conventional oral appliances are not easily adjustable by the SAS sufferer and do not allow for the natural movement of the jaws of the SAS sufferer while sleeping.

### SUMMARY

The present invention provides an oral appliance that is easily adjustable by the SAS sufferer and allows for the natural movement of the jaws of the SAS sufferer while sleeping.

The present invention provides an oral appliance which includes a first jaw retainer piece, a second jaw retainer piece, and a holding member mounted on a front-facing portion of the first jaw retainer piece, wherein the holding member has an opening for receiving a rod. Further, the present invention provides that the leading (distal) end of the rod is slidably inserted into the opening of the holding member and a trailing end of the rod is mounted on a front-facing portion of the second jaw retainer piece. The oral appliance further includes a restrictor that restricts sliding of the rod relative to the holding member within the opening.

According to the present invention, the holding member can be a tubularly shaped or a ring shaped. Further, according to the present invention, the restrictor can be formed as part of the holding member to which the leading end of the rod abuts, thereby restricting the sliding of the rod relative to the holding member. Further still, according to the present invention, the restrictor can be a ring that encircles the rod and is position at the trailing end of the rod to which the holding member abuts, thereby restricting the sliding of the rod relative to the holding member. Moreover, according to the present invention, the restrictor can be an element that engages the leading end of the rod to which the holding member abuts, thereby restricting the sliding of the rod relative to the holding member.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

In the detailed description that follows, embodiments are described as illustrations only since various changes and modifications will become apparent to those skilled in the art from the following detailed description. The use of the same reference numbers in different figures indicates similar or identical items.

FIG. 1 shows an exemplary oral appliance after being assembled in accordance with at least some embodiments described herein;

FIG. 2 shows a top portion of an exemplary holding member mounted on a front-facing portion of a lower jaw retainer piece in accordance with at least some embodiments described herein;

FIG. 3 shows a top portion of an exemplary rod mounted on a front-facing portion of an upper jaw retainer piece in accordance with at least some embodiments described herein;

FIGS. 4A and 4B show an exemplary holding member and rod in accordance with at least some embodiments described herein;

FIG. 5 shows an exemplary rod in accordance with at least some embodiments described herein;

FIGS. 6A and 6B show an exemplary oral appliance while adjusting a lower jaw position in accordance with at least some embodiments described herein;

FIG. 7A and 7B show a cross sectional view of an exemplary oral appliance while adjusting a lower jaw position in accordance with at least some embodiments described herein;

FIG. 8 shows a cross sectional view of an exemplary oral appliance in accordance with at least some embodiments described herein;

FIG. 9 shows a cross sectional view of an exemplary oral appliance in accordance with at least some embodiments described herein;

FIG. 10 shows an exemplary oral appliance after being assembled in accordance with at least some embodiments described herein;

FIG. 11A shows a top portion of an exemplary holding member mounted on a front-facing portion of a lower jaw retainer piece in accordance with at least some embodiments described herein;

FIG. 11B shows a front portion of an exemplary holding member mounted on a front-facing portion of a lower jaw retainer piece in accordance with at least some embodiments described herein;

FIG. 12A shows a top portion of an exemplary rod mounted on a front-facing portion of an upper jaw retainer piece in accordance with at least some embodiments described herein;

FIG. 12B shows a bottom portion of an exemplary rod mounted on a front-facing portion of an upper jaw retainer piece in accordance with at least some embodiments described herein;

FIGS. 13A and 13B show an exemplary holding member and rod in accordance with at least some embodiments described herein;

FIG. 14 shows an exemplary rod and restrictor in accordance with at least some embodiments described herein;

FIGS. 15A and 15B show an exemplary oral appliance while adjusting a lower jaw position in accordance with at least some embodiments described herein;

FIG. 16A shows a cross sectional view of an exemplary oral appliance in accordance with at least some embodiments described herein; and

FIG. 16B shows a cross sectional view of an exemplary oral appliance in accordance with at least some embodiments described herein.

### DETAILED DESCRIPTION

In the following detailed description, reference is made to the accompanying drawings, which form a part of the description. In the drawings, similar symbols typically identify similar components, unless context dictates otherwise. Furthermore, unless otherwise noted, the description of each successive drawing may reference features from one or more of the previous drawings to provide clearer context and a more substantive explanation of the current example embodiment. Still, the example embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein and illustrated in the drawings, may be arranged, substituted, combined, separated, and designed in a wide variety of different configurations, all of which are explicitly contemplated herein.

Described herein are related to an oral appliance intended to be placed in a user's mouth for preventing and treating sleep apnea syndrome.

In the present description, the following terms may be used, in addition to their accepted meaning, as follows:

“Forward” and “backward” directions may refer to respectively, a direction toward the front of a user wearing the oral appliance (a direction toward a mouth opening as viewed from a throat in the oral cavity) and a direction toward the rear (in a direction from the mouth opening to the throat in the oral cavity); the “right and left direction” or “rightward and leftward” refers to from the center of the upper jaw of the user wearing the oral appliance as a reference, to the right and left directions (a direction toward a right cheek and a direction toward a left cheek); and “upward and downward direction” refers to from the center of the upper jaw of the user wearing the oral appliance as a reference, to a direction towards upside (in a direction from the center of the upper jaw to a nose) and a direction towards downside (in a direction from the center of the upper jaw to the lower jaw).

A “longitudinal direction” may refer to the axial direction of a rod or a holding member and back to front, and a “lateral direction” may refer to a side to side direction.

A “front-facing portion” of a first jaw retainer piece and a second jaw retainer piece may refer to, respectively, a front face portion of the first jaw retainer piece and a front face portion of the second jaw retainer.

In accordance with some embodiments, the oral appliance comprises a first jaw retainer piece; a second jaw retainer piece; a holding member mounted on a front-facing portion of the first jaw retainer piece, the holding member having an opening for receiving a rod; the rod having a leading end slidably inserted into the opening of the holding member and a trailing end mounted on a front-facing portion of the second jaw retainer piece; and a restrictor that restricts sliding of the rod relative to the holding member.

It is important to provide effective oral appliance in maintaining the mandible in a protruded position to improve airway patency, they often result in undesirable side effects. The need has therefore arisen for an oral appliance for treatment of snoring and sleep apnea which will effectively maintain the mandible in a preferred anterior position, but which will also allow a limited degree of forward or lateral excursion of the mandible relative to the upper jaw to avoid discomfort to the temporomandibular joint and related muscles and ligaments. The embodiments described herein improve user experience by allow a predetermined degree of lateral movement or forward movement of mandible in the protruded position, while still maintaining acceptable airway patency effectively by maintaining the mandible in a preferred position.

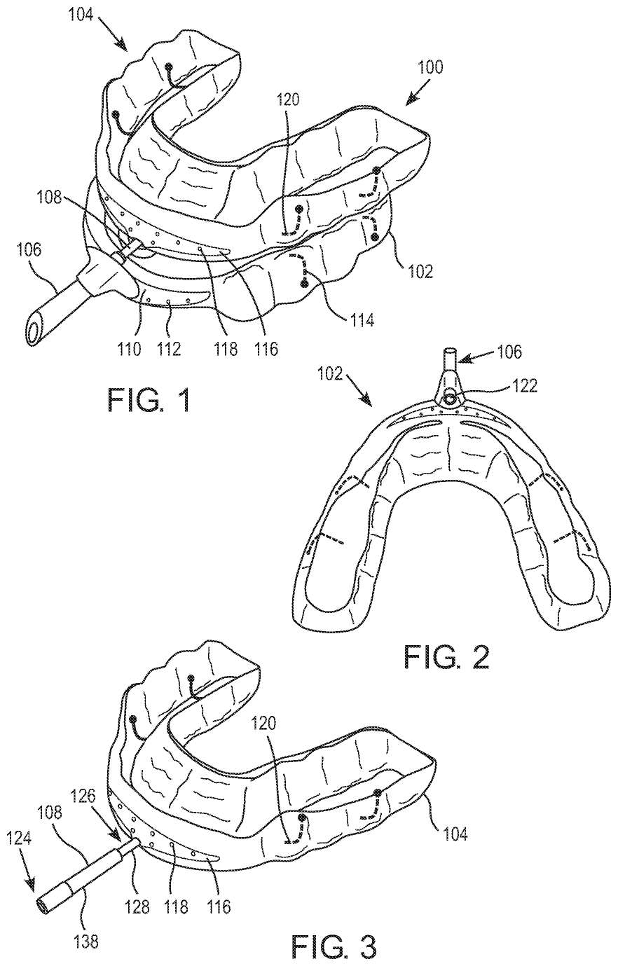

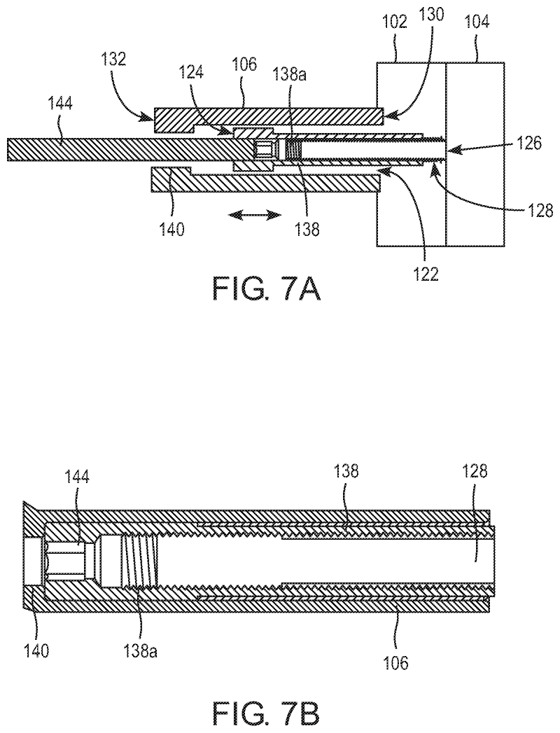

FIG. 1 shows an exemplary oral appliance after being assembled in accordance with at least some embodiments described herein. As illustrated in FIG. 1, the exemplary oral appliance 100 includes, at least a first jaw retainer piece, a second jaw retainer piece, a holding member 106, and a rod 108. The first jaw retainer piece may refer to a lower jaw retainer piece 102 and the second jaw retainer piece may refer to an upper jaw retainer piece 104. The holding member 106 is mounted on a front-facing portion of the lower jaw retainer piece 102, and has an opening 122 (as shown in FIG. 2) for receiving a rod 108. The holding member 106 may be tubularly shaped. The oral appliance 100 may include a restrictor 140 (as shown in FIGS. 7A and B) that restricts sliding of the rod 108 relative to the holding member 106. The holding member 106 is mounted on or attached to the front-facing portion of the lower jaw retainer piece 102 via a first mounting unit 110. The first mounting unit 110 includes a first plurality of through-holes 112 which allow for the infiltration of the material that forms the lower jaw retainer piece 102 for positional stability. The lower jaw retainer piece 102 includes a first fixing member 114 which is a dental wire (or a plurality of metallic dental wires) for fixing the user's teeth more firmly in place in the lower jaw retainer piece 102.

One end of the rod 108 is mounted on a front-facing portion of the upper jaw retainer piece 104, and the other end of the rod 108 is slidably inserted into the opening 122 of the holding member 106 along a longitudinal direction of the holding member 106. The one end of the rod 108 is mounted on the front-facing portion of the upper jaw retainer piece 104 via a second mounting unit 116. The second mounting unit 116 includes a second plurality of through-holes 118 which allow for the infiltration of the material that forms the upper jaw retainer piece 104 for positional stability. The upper jaw retainer piece 104 include a second fixing member 120 which is a dental wire (or a plurality of metallic dental wires) for fixing the user's teeth more firmly in place in the upper jaw retainer piece 104.

FIG. 2 shows a top portion of an exemplary holding member mounted on a front-facing portion of a lower jaw retainer piece 102 in accordance with at least some embodiments described herein. A lower jaw retainer piece 102 is shaped and adapted to engage the mandibular dentition of the user, and the lower jaw retainer piece 102 is open towards a mandibular. The size and shape of the lower jaw retainer piece 102 can be customized to suit the anatomy of each user. The lower jaw retainer piece 102 may include a first fixing member 114 (as shown in FIG. 1) which is a metallic (e.g. stainless steel) dental wire (or a plurality of metallic dental wires) to ensure that the lower jaw retainer piece 102 securely engages the user's teeth.

The holding member 106 may be an elongated hollow and tubularly shaped member which is longitudinally perpendicular to a front-facing portion of the lower jaw retainer piece 102. The holding member 106 has a proximal end which is mounted on or attached to the front-facing portion of the lower jaw retainer piece 102 by the first mounting unit 110, and a distal end extending along a longitudinal axis in a forward direction. The holding member 106 has an opening 122 for receiving the rod 108 at the proximal end and another opening at the distal end 132. The opening 122 and the other opening are connected in a tubular shape body of the holding member 106 along the longitudinal axis. The opening 122 is adapted to receive the rod 108 from the front-facing portion of the lower jaw retainer piece 102 towards the distal end of the holding member along the longitudinal axis of the holding member 106.

FIG. 3 shows a top portion of an exemplary rod mounted on a front-facing portion of an upper jaw retainer piece in accordance with at least some embodiments described herein. An upper jaw retainer piece 104 is shaped and adapted to engage the maxillary dentition of the user, and the upper jaw retainer piece 104 is open towards a maxillary. The size and shape of the upper jaw retainer piece 104 can be customized to suit the anatomy of each user. The upper jaw retainer piece 104 may include a second fixing member 120 which may be a metallic (e.g. stainless steel) dental wire (or a plurality of metallic dental wires) to ensure that the upper jaw retainer piece 104 securely engages the user's teeth.

The rod 108 is an elongated member that includes a trailing end 126 and a leading end 124. The trailing end 126 of the rod 108 is mounted on or attached to the front-facing portion of the upper jaw retainer piece 104 by the second mounting unit 116, and the leading end 124 is extending along a longitudinal axis in a forward direction. When the rod 108 is assembled with the holding member 106 in accordance with at least some embodiments described herein, the leading end 124 of the rod 108 may be slidably inserted into the opening 122 of the holding member 106 along the longitudinal axis of the holding member 106 in a forward direction.

The rod 108 may comprise two segments including an inner segment and outer segment. The inner segment of the rod 108 may be a screw portion 128 that extends from the upper jaw retainer piece 104 and the outer segment of the rod 108 may be a screw-adjustable portion 138 that engages with the screw portion 128 to move in forward and backward directions. As per FIGS. 7A and 7B, the screw-adjustable portion 138 (the outer segment) may be a hollow cylindrical shape and holds the screw portion 128 (the inner segment) therein. The screw-adjustable portion 138 has an open end at the leading end 124 and another open end at opposite side, and the open end and another open end form an elongated opening through rod 108 along its longitudinal axis. One end of the screw portion 128 is mounted on or attached to the front-facing portion of the upper jaw retainer piece 104 by the second mounting unit 116 and is the trailing end 126 of the rod 108. Another end of the screw portion 128 is positioned inside the screw-adjustable portion 138 and is extended towards the leading end 124 of the rod 108. The screw portion 128 has external threads thereon, which are male threads. The another end of the screw-adjustable portion 138 is formed to receive a shaft of a screwdriver which can be used to adjust the positioning of the screw-adjustable portion 138 relative to the trailing end 126 of the screw portion 128. The inner surface of the screw-adjustable portion 138 includes a set of internal threads 138a (shown in FIGS. 7A and 7B) which are female threads, and threadingly engaged with the screw portion 128.

The screw-adjustable portion 138 is movable longitudinally relative to the screw portion 128 between a retracted position and an extended position so as to effect telescopic transition of the screw-adjustable portion 138 with respect to the screw portion 128. When the screw-adjustable portion 138 is screwed by the screw driver, the screw-adjustable portion 138 is moving forward relative to the trailing end 126 of the screw portion 128, and the screw-adjustable portion 138 moves to the extended position. The screw portion 128, which was previously positioned inside of the screw-adjustable portion 138, is coming out from the screw-adjustable portion 138, and a total length of the rod 108 is extended and getting longer. When the screw-adjustable portion 138 is screwed by the screw driver, the set of internal threads 138a in the inner surface of the screw-adjustable portion 138 threadingly engages with the screw portion 128 to move in a forward direction. Since one end (a trailing end 126) of the screw portion 128 is mounted on or attached to the front-facing portion of the upper jaw retainer piece 104, as the screw portion 128 is coming out from the screw-adjustable portion 138, a position of the screw-adjustable portion 138 moves in the forward direction.

When the screw-adjustable portion 138 is unscrewed (or reverse screwed) by the screw driver, the screw-adjustable portion 138 is moving backward relative to the trailing end 126 of the screw portion 128, and the screw-adjustable portion 138 moves towards the retracted position. The screw portion 128, which was previously positioned outside of the screw-adjustable portion 138, is coming back to the screw-adjustable portion 138, and the extended total length of the rod 108 is retracted and shortened. Accordingly, a relative position of the screw-adjustable portion 138 moves in a backward direction. The total length of the rod 108 or a relative position of the screw-adjustable portion 138 is adjustable by screwing or unscrewing the screw-adjustable portion 138. The screw-adjustable portion 138 may have a larger outer diameter at the leading end 124 side than opposite end side thereof.

In accordance with some implementations, the lower jaw retainer piece 102 and the upper jaw retainer piece 104 may be made of, but not limited to, for example, acrylic resin. The lower jaw retainer piece 102 and the upper jaw retainer piece 104 are preferably made of a single hard material having a flexural modulus of 2,000 MPa or more and 3,000 MPa or less, or combination of a soft material having a flexural modulus of 10 MPa or more and 300 MPa or lower and a hard material having a flexural modulus of 1,000 MPa or more and 3,000 MPa or less.

The lower jaw retainer piece 102 and the upper jaw retainer piece 104 may also be made of relatively soft material having a tensile strength of 150 N or more and less than 2,000 N, particularly 150 N or more and 500 N or less. The lower jaw retainer piece 102 and the upper jaw retainer piece 104 made of the aforementioned materials have high conformity to the teeth when they are worn by the user. The tensile strength refers to the strength at which an oral appliance (3-mm thick) fabricated with a Nissin standard model tears in a tensile testing conducted in the molar direction (backward on the row of teeth) with a hole 1.5 mm in diameter bored at the sixth tooth in the lower jaw retainer piece and the upper jaw retainer piece of the oral appliance.

Materials having a tensile strength of 150 N or more and less than 2,000 N include, for example, olefin-based resin, polyester-based resin, urethane-based resin, polyamide-based resin, or acrylic rubber resin, where the olefin-based resin is preferable inter alia. The olefin-based resin is a polymer made by homopolymerization of olefin or a copolymer of olefin and another monomer. Olefin with a carbon number of 2 to 6 containing ethylene, propylene, butene, methylpentene, and hexene is preferred. The another monomer may be for example, vinyl acetate.

Preferably, the olefin-based resin is polyethylene (PE), polyethylene-based resin, polypropylene (PP), polypropylene-based resin, and ethylene-vinyl acetate copolymer (EVA), for example, and more preferably, is polyethylene (PE), polyethylene-based resin, polypropylene (PP), or polypropylene-based resin.

Polyester-based resin is a polycondensate of polyvalent carboxylic acid (dicarboxylic acid) and polyalcohol (diol), for example, polyethyleneterephthalate (PET). Urethane-based resin is a polycondensate of a compound having an isocyanate group and a compound having a hydroxy group, for example, is thermoplastic polyurethane (TPU).

Polyamide-based resin is a polymer formed by binding of many monomers by amide bond, for example, nylon, para-amide, and meta-amide. Acrylic rubber resin consists mainly of acrylic rubber, for example, is a block copolymer of methyl methacrylate and butyl acrylate.

Material having a tensile strength of 150 N or more and less than 2,000 N may be a commercially available material, for example, F327, a polypropylene resin manufactured by Prime Polymer Co., Ltd.

In accordance with some implementations, the rod 108 and the holding member 106 may be made of, but not limited to, titan, or alloys containing titan, iron or steel such as stainless gold, silver, platinum, cobalt or chromium, and a hard material having a flexural modulus of 2,000 MPa or more and 3,000 MPa (such as polycarbonate).

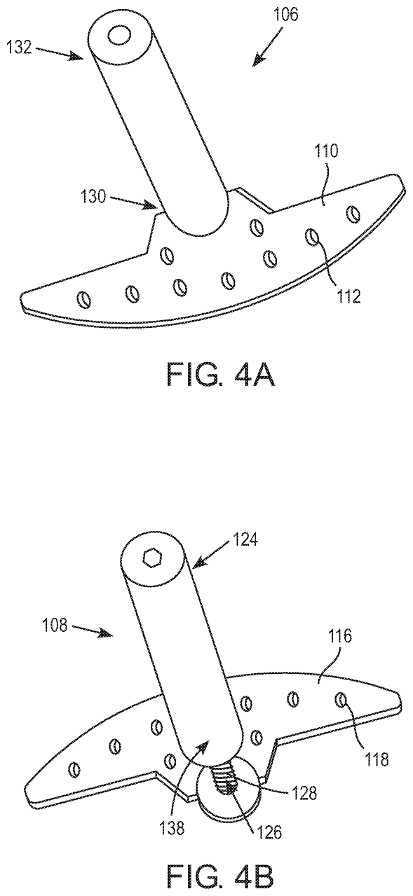

FIG. 4A shows an exemplary holding member and FIG. 4B shows an exemplary rod in accordance with at least some embodiments described herein. The holding member 106 has a proximal end 130 which is to be mounted on or attached to the front-facing portion of the lower jaw retainer piece 102 by the first mounting unit 110, and a distal end 132 extending along a longitudinal axis in a forward direction. The proximal end 130 of the holding member 106 is attached to the first mounting unit 110 that is adapted to be mounted on or attached to the front-facing portion of the lower jaw retainer piece 102. The first mounting unit 110 is a thin plate-like member that may be made of material which may be used for forming the rod 108 or the holding member 106. The first mounting unit 110 may be formed in a curved shape along the shape of a front-facing portion of the lower jaw retainer piece 102. The first mounting unit 110 may have a first plurality of through-holes 112 for facilitating the fixation of the first mounting unit 110 to the lower jaw retainer piece 102. The first mounting unit 110 may be mounted on the front-facing portion of the lower jaw retainer piece 102 using a resin material which may be used for forming the lower jaw retainer piece 102. The resin material may pass through the first plurality of through-holes 112 and attaching or fixing the position of the first mounting unit 110 to the lower jaw retainer piece 102 to enhance the fixing strength each other.

The rod 108 is an elongated member that includes a trailing end 126 and a leading end 124. The trailing end 126 of the rod 108 is to be mounted on or attached to the front-facing portion of the upper jaw retainer piece 104 by the second mounting unit 116, and the leading end 124 is extending along a longitudinal axis in a forward direction. The trailing end 126 of the rod 108 is attached to the second mounting unit 116 that is adapted to be mounted on or attached to the front-facing portion of the upper jaw retainer piece 104. The second mounting unit 116 is a thin plate-like member that may be made of material which may be used for forming the rod 108 or the holding member 106. The second mounting unit 116 may be formed in a curved shape along the shape of a front-facing portion of the upper jaw retainer piece 104. The second mounting unit 116 may have a second plurality of through-holes 118 for facilitating the fixation of the second mounting unit 116 to the upper jaw retainer piece 104. The second mounting unit 116 may be mounted on or attached to the front-facing portion of the upper jaw retainer piece 104 using a resin material which may be used for forming the upper jaw retainer piece 104. The resin material may pass through the second plurality of through-holes 118 and attach or fix the position of the second mounting unit 116 to the upper jaw retainer piece 104 and enhance the fixing strength each other.

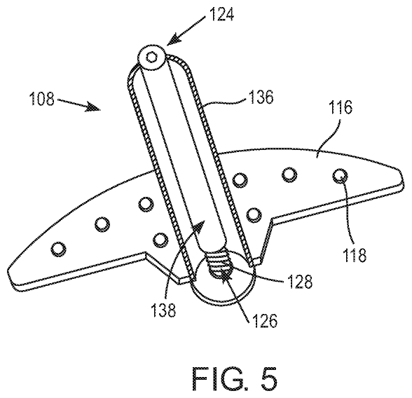

FIG. 5 shows an exemplary rod in accordance with at least some embodiments described herein. In FIG. 5, the rod 108 may optionally include a protective cover 136 that covers at least a part of the rod 108. The protective cover 136 may have a substantially hollow semi-cylindrical shape defining a rounded inner surface. One end of the protective cover 136 is mounted on the second mounting unit 116 and located above the rod 108, and the other end of the protective cover 136 is extended along a longitudinal axis of the rod 108 and parallel to the rod 108. The rounded inner surface of the protective cover 136 faces the rod 108, and a gap exists between the protective cover 136 and the rod 108. After the rod 108 is slidably inserted into the opening 122 of the holding member 106 and assembled with the holding member 106, the holding member 106 is positioned at the gap between the protective cover 136 and the rod 108, and the protective cover 136 covers at least a part of the holding member 106 in which the rod 108 is inserted. Therefore, after assembly of the rod 108 and the holding member 106, the rounded inner surface of the protective cover 136 faces the holding member 106.

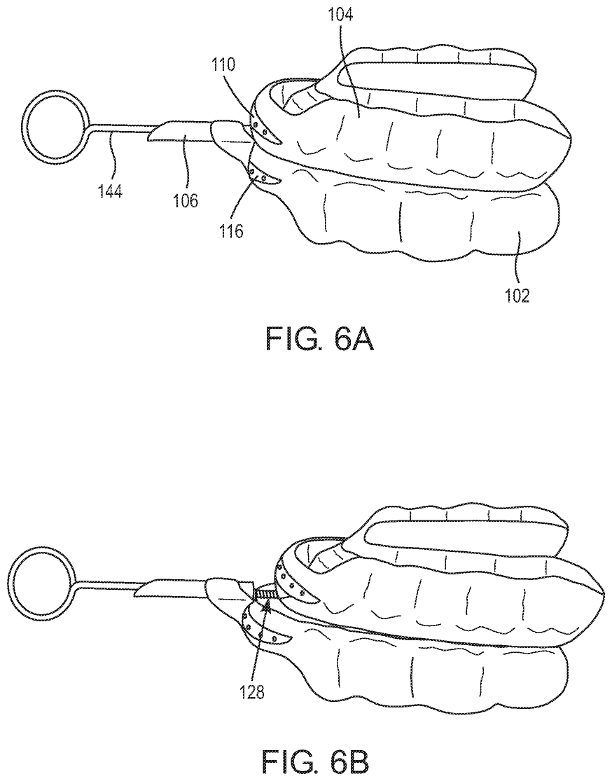

FIGS. 6A and 6B show an exemplary oral appliance while adjusting a lower jaw position in accordance with at least some embodiments described herein. The assembled oral appliance 100 worn by the user is illustrated in FIGS. 6A and 6B. To assemble the oral appliance 100, as described above, the leading end 124 of the rod 108 is slidably inserted into the opening 122 of the holding member 106 from the front-facing portion of the lower jaw retainer piece 102 towards a forward direction along a longitudinal axis of the holding member 106. The trailing end 126 of the rod 108 is mounted on the front-facing portion of the upper jaw retainer piece 104 via the second mounting unit 116. The lower jaw position of the user may be adjusted after or before the user wears the assembled oral appliance.

FIG. 6A shows that the position of the lower jaw retainer piece 102 and the position of the upper jaw retainer piece 104 are initially same, and the lower jaw retainer piece 102 has not moved forward yet relative to the upper jaw retainer piece 104. The position of the screw-adjustable portion 138 of the rod 108 is adjusted in forward and backward directions to adjust an abutment position of the rod 108 relative to a restrictor (as shown in FIG. 7). As described above, a relative position of the screw-adjustable portion 138 of the rod 108 is adjusted by screwing or unscrewing the screw-adjustable portion 138. The rod 108 comprises the screw portion 128 that extends from the upper jaw retainer piece 104 and the screw-adjustable portion 138 that engages with the screw portion 128 to move in forward and backward directions. In FIG. 6A, the screw-adjustable portion 138 is at a fully retracted position and the screw portion 128 is located within the screw-adjustable portion 138.

As described above regarding FIG. 3, the screw-adjustable portion 138 (the outer segment) may be a hollow cylindrical shape and engages the screw portion 128 (the inner segment) therein. The screw-adjustable portion 138 has an open end at the leading end 124 and another open end at opposite side, and the open end and another open end form an elongated opening along a longitudinal axis of the screw-adjustable portion 138. One end of the screw portion 128 is mounted on or attached to the front-facing portion of the upper jaw retainer piece 104 and is the trailing end 126 of the rod 108. Another end of the screw portion 128 is positioned inside the screw-adjustable portion 138 and extended towards the leading end 124 of the rod 108. The another end of the screw-adjustable portion 138 is formed to receive a shaft of a screwdriver 144 which can be used to adjust the positioning of the screw-adjustable portion 138 relative to the trailing end 126 of the screw portion 128. The inner surface of the screw-adjustable portion 138 includes a set of internal threads 138a which is female threads, and threadingly engages with the screw portion 128. As shown in FIG. 2, the holding member 106 has an opening 122 for receiving the rod 108 at the proximal end 130 and another opening at the distal end 132. The opening 122 and the another opening are connected through a tubular shape body of the holding member 106 along its longitudinal axis. The opening 122 is adapted to receive the rod 108 from the front-facing portion of the lower jaw retainer piece 102 towards the distal end 132 of the holding member 106 along its longitudinal axis. Thus, after the rod 108 is inserted into and assembled with the holding member 106, the screwdriver 144 may be inserted into the another opening at the distal end 132 of the holding member 106. Then, the another end of the screw-adjustable portion 138 receives a shaft of a screwdriver 144 which can be used to adjust the positioning of the screw-adjustable portion 138 relative to the trailing end 126 of the screw portion 128.

After the rod 108 is inserted into the holding member 106 and assembled, the holding member 106 holds the screw-adjustable portion 138. The screw-adjustable portion 138 is the outer segment of the rod 108, and engages the screw portion 128 (the inner segment) therein. As the screw-adjustable portion 138 is screwed by the screw driver, the screw-adjustable portion 138 is moving forward relative to the trailing end 126 of the screw portion 128, and the screw-adjustable portion 138 moves to the extended position. The set of internal threads 138a in the inner surface of the screw-adjustable portion 138 threadingly engages with the screw portion 128 and the screw-adjustable portion 138 moves in a forward direction. Therefore, a total length of the rod 108 is extended and getting longer. As the position of the screw-adjustable portion 138 moves in a forward direction, the position of the holding member 106 that holds the screw-adjustable portion 138 of the rod 108 also moves in the forward direction. Accordingly, the position of the lower jaw retainer piece 102 which is attached to the holding member 106 also moves in the forward direction. On the other hand, as one end of the screw portion 128 (i.e. the trailing end 126 of the rod 108) is mounted on the front-facing portion of the upper jaw retainer piece 104, the position of the upper jaw retainer piece 104 which is attached to the rod 108 maintains same position. Thereby, the lower jaw retainer piece 102 is positioned forward relative to the upper jaw retainer piece 104. In particular, FIG. 6B shows that the position of the lower jaw retainer piece 102 and the position of the upper jaw retainer piece 104 are different after screwing, and the lower jaw retainer piece 102 has moved in a forward direction relative to the upper jaw retainer piece 104. In FIG. 6B, the screw-adjustable portion 138 is at an extended position and a portion of the screw portion 128 is located outside of the screw-adjustable portion 138.

When the screw-adjustable portion 138 is unscrewed (or reverse screwed) by the screwdriver 144, the screw-adjustable portion 138 is moving backward relative to the trailing end 126 of the screw portion 128, and the screw-adjustable portion 138 moves towards the retracted position. The screw portion 128, which was previously positioned outside of the screw-adjustable portion 138, is coming back to the screw-adjustable portion 138, and the extended total length of the rod 108 is retracted. Accordingly, the position of the screw-adjustable portion 138 moves in a backward direction relative to the previous position. As the position of the screw-adjustable portion 138 moves in a backward direction, the position of the holding member 106 that holds the screw-adjustable portion 138 of the rod 108 also moves in a backward direction. Accordingly, the position of the lower jaw retainer piece 102 which is attached to the holding member 106 also moves in a backward direction.

By positioning the lower jaw retainer piece 102 forward relative to the upper jaw retainer piece 104, the oral appliance 100 can restrict the displacement of the lower jaw of the user to the backward direction, thereby preventing the constriction of the airway, a breathing stop or low respiration during sleep. As the oral appliance 100 adjusts the amount of advancement of the lower jaw retainer piece 102 relative to the upper jaw retainer piece 104, the lower jaw moves forward relative to the upper jaw by the adjusted amount of the advancement. Therefore, it is possible to make the user's airway less likely to be narrowed.

FIGS. 7A and 7B show a cross sectional view of an exemplary oral appliance while adjusting a lower jaw position in accordance with at least some embodiments described herein. In FIG. 7A, the holding member 106, which is tubularly shaped, includes a restrictor 140 that restricts sliding of the rod 108 relative to the holding member 106. The holding member 106 has a proximal end 130, a distal end 132, and a middle portion located between the proximal end 130 and the distal end 132. For example, the restrictor 140 is a ring-shaped protrusion portion provided on an inner circumference of the holding member 106 at a distal end 132, and is a cylindrical portion with a smaller inner diameter. The inner diameter of the restrictor 140 is smaller than the inner diameter of the proximal end 130 or the middle portion of the holding member 106. Accordingly, the restrictor 140 makes a diameter of the another opening at the distal end 132 smaller than a diameter of the opening 122 at the proximal end 130 or the middle portion of the holding member 106.

Further, the screw-adjustable portion 138, which is a hollow cylindrical shape that engages the screw portion 128 therein, may have an outer diameter at the leading end 124 side larger than an outer diameter at the opposite end side. The inner diameter of the restrictor 140 (i.e. a diameter of the another opening at the distal end 132) may be large enough to receive a screwdriver 144 but may be smaller than an outer diameter of the screw-adjustable portion 138 at the leading end 124 side. Therefore, even after setting an abutment position of the rod 108 relative to the restrictor 140 via screwing (i.e. a position of the lower jaw retainer piece 102 relative to a position of the upper jaw retainer piece 104), the leading end 124 of the rod 108 can slide forward and backward within the holding member 106. However, the leading end 124 of the rod 108 cannot pass beyond the restrictor 140 in a forward direction, and the restrictor 140 restricts sliding of the rod relative within the holding member. Similarly, the distal end 132 of the holding member 106 can move in a forward and backward directions relative to the rod 108 but cannot pass over the leading end 124 of the rod 108 in a backward direction. FIG. 7B shows a partial cross sectional view of an exemplary oral appliance in accordance with at least some embodiments described herein. In particular, FIG. 7B illustrates the inner surface of the screw-adjustable portion 138a which includes a set of threads which are female threads.

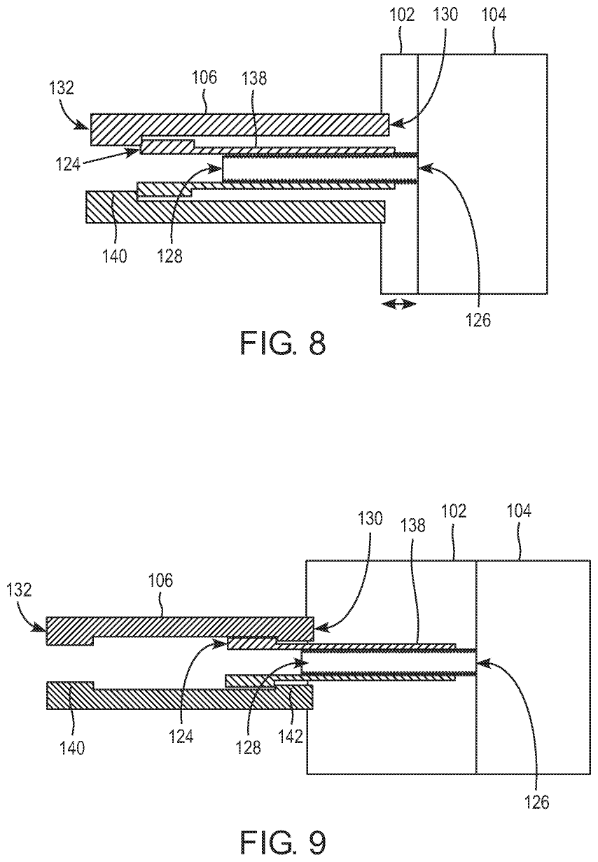

FIG. 8 shows a cross sectional view of an exemplary oral appliance in accordance with at least some embodiments described herein. In FIG. 8, the restrictor 140 abuts against the rod 108 and restricts a forward movement of the rod 108 within the holding member 106. Further, the restrictor 140 restricts a backward movement of the holding member 106 relative to the rod 108. Therefore, the restrictor 140 prevents the rod 108 from sliding too much in a forward direction within the holding member 106, and further prevents the holding member 106 from sliding in a backward direction relative to the rod 108. Still, the holding member 106 can move in a forward direction. If the holding member 106 does not have the restrictor 140, the rod 108 further moves to a forward direction, and the holding member 106 moves backward, and therefore, the upper jaw retainer piece 104 attached to the rod 108 moves to a forward direction and the lower jaw retainer piece 102 attached to the holding member 106 moves can move backward. The restrictor 140 restricts the displacement of the lower jaw of the user to the backward direction during sleeping and therefore an oral appliance 100 allows the user to maintain the position of the lower jaw forward relative to the upper jaw. At the same time, the restrictor 140 does not restrict the user to move the lower jaw forward relative to the upper jaw when the oral appliance 100 is worn by the user during sleeping. Therefore, the restrictor 140 sets a predetermined degree of a forward and backward movement of mandible within the protruded position, while still maintaining acceptable airway patency effectively by maintaining the mandible in a preferred protruded position. Therefore, the oral appliance 100 allows the user to move the lower jaw forward within a predetermined degree during sleeping, and provides more flexibility, comfortable use, and ease of use of the oral appliance 100 to the user who worn the oral appliance. Thereby, the oral appliance 100 provides great user experience without detriment the effectiveness and function of mandibular advancement.

In accordance with some implementations, the opening 122 of the holding member 108 has an ellipse shape, an oval shape, an egg shape, a rectangle shape, or any oblong shape. The term “oblong” may include a structure having a dimension, especially one of two perpendicular dimensions, such as, for example, width or length, that is greater than another and includes shapes such as rectangles, ovals, ellipses, and any other elongated shapes. In particular, the opening 122 has an oblong shape having a width along the right and left directions, and the width is larger than a length of the oblong shape that is perpendicular to the width. The larger width of the opening 122 enables more free movement of the rod 108 along the right and left directions within the holding member 106, and more free movement of the holding member 106 rightward and leftward relative to the rod 108. For example, the opening 122 has an ellipse shape having a major axis and a minor axis. The major axis, which is the longest diameter of an ellipse shape is along the right and left directions, and the minor axis, which is the shortest diameter of the ellipse is along the upward and downward directions. Therefore, the rod 108 is allowed to more freely move rightward and leftward along the major axis of the ellipse shaped opening 122 within the holding member 106, and the holding member 106 is also allowed to move rightward and leftward relative to the rod 108. Therefore, the oral appliance 100 allows the user to move the lower jaw or upper jaw rightward and leftward relative to each other during sleeping. The embodiments described herein improve user experience by allow a predetermined degree of lateral movement of mandible and provide more flexibility, comfortable use, and ease of use of the oral appliance 100 to the user who wears the oral appliance. Accordingly, the oral appliance 100 provides great user experience without detriment the effectiveness and function of mandibular advancement.

FIG. 9 shows a cross sectional view of an exemplary oral appliance in accordance with at least some embodiments described herein. In FIG. 9, the oral appliance 100 includes another restrictor 142 at opposite side of the restrictor 140. The another restrictor 142 prevents the rod 108 from being fully withdrawn from the holding member 106. The holding member 106 has a proximal end 130, a distal end 132, and a middle portion located between the proximal end 130 and the distal end 132. For example, the another restrictor 142 is a ring-shaped protrusion portion provided on an inner circumference of the holding member 106 at the proximal end 130, and is a cylindrical portion with a smaller inner diameter. The inner diameter of the another restrictor 142 is smaller than the inner diameter of the middle portion of the holding member 106. The inner diameter of the another restrictor 142 may be substantially same as the inner diameter of the restrictor 140. The another restrictor 142 abuts against the rod 108 and restricts the rod 108 from being fully withdrawn from the holding member 106 in a backward direction. Further, the another restrictor 142 restricts the holding member 106 from being fully withdrawn from the rod 108 in a forward direction.

The another restrictor 142 may be manufactured as a ring-shaped protrusion portion provided on an inner circumference of the holding member 106 at the proximal end 130, or alternatively, may be a separate supporting member. The separate supporting member may be inserted after assembly of the rod 108 and holding member 106 (i.e. after the rod 108 is inserted into the opening 122 of the holding member 106) and fixed to the inside of the holding member 106 at the proximal end 130 to prevent the rod 108 from being fully withdrawn from the rod 108.

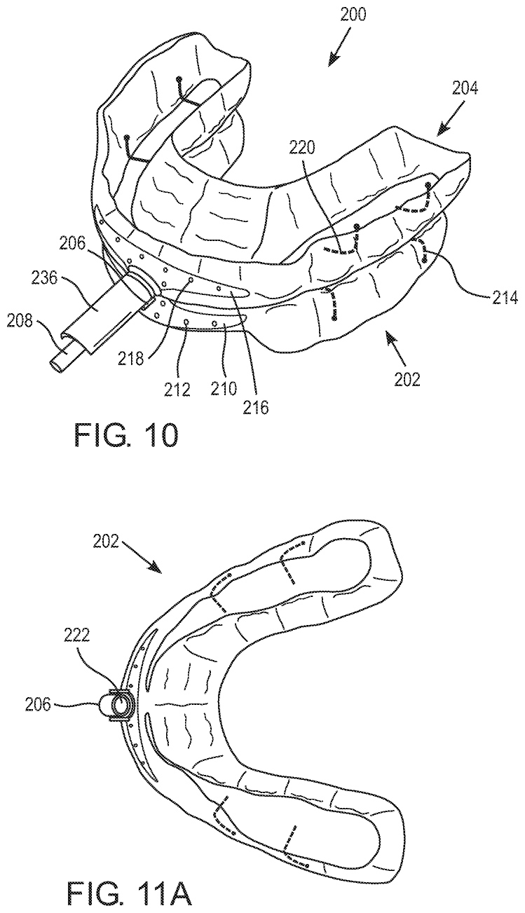

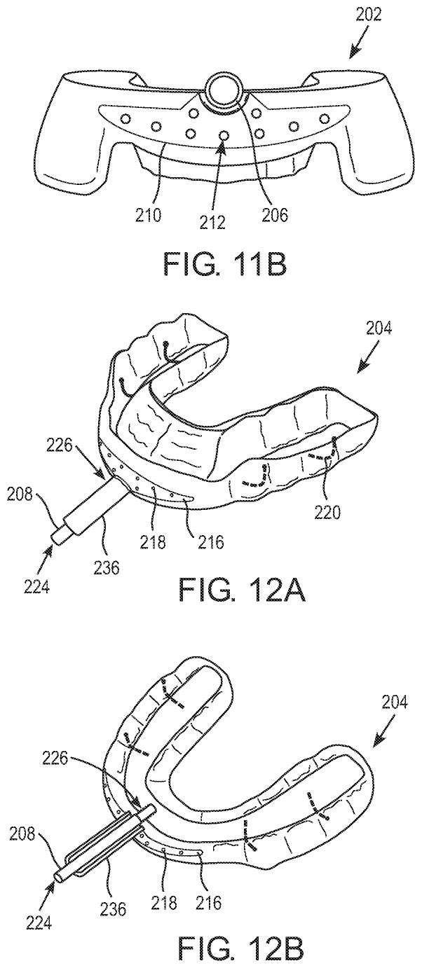

FIG. 10 shows an exemplary oral appliance after being assembled in accordance with at least some embodiments described herein. As depicted, another exemplary oral appliance 200 includes, at least a first jaw retainer piece, a second jaw retainer piece, a holding member 206, and a rod 208. The first jaw retainer piece may refer to a lower jaw retainer piece 202 and the second jaw retainer piece may refer to an upper jaw retainer piece 204. The holding member 206 is mounted on the front-facing portion of the lower jaw retainer piece 202, and has an opening 222 (as shown in FIG. 11A) for receiving a rod 208. The holding member 206 may have a ring shape. The oral appliance 200 may include a restrictor 240, or another restrictor 242 (as shown in FIG. 14), that restricts sliding of the rod 208 relative to the holding member 206. The holding member 206 may be mounted on or attached to a front-facing portion of the lower jaw retainer piece 202 via a first mounting unit 210. The first mounting unit 210 includes a first plurality of through-holes 212 which allow for the infiltration of the material that forms the lower jaw retainer piece 202 for positional stability. The lower jaw retainer piece 202 includes a first fixing member 214 which is a metallic (e.g. stainless steel) dental wire (or a plurality of metallic dental wires) for fixing the teeth more firmly in place in the lower jaw retainer piece 202.

One end of the rod 208 is mounted on the front-facing portion of the upper jaw retainer piece 204, and the other end of the rod 208 is slidably inserted into the opening 222 of the holding member 206 in a forward direction. The one end of the rod 208 is mounted on the front-facing portion of the upper jaw retainer piece 204 via a second mounting unit 216. The second mounting unit 216 includes a second plurality of through-holes 218 which allow for the infiltration of the material that forms the upper jaw retainer piece 204 for positional stability. The upper jaw retainer piece include a second fixing member 220 which is a metallic (e.g. stainless steel) dental wire (or a plurality of metallic dental wires) for fixing the teeth more firmly in place in the upper jaw retainer piece 204.

FIG. 11A shows a top portion of an exemplary holding member mounted on a front-facing portion of a lower jaw retainer piece in accordance with at least some embodiments described herein. A lower jaw retainer piece 202 is shaped and adapted to engage the mandibular dentition of a user, and the lower jaw retainer piece 202 is open towards a mandibular. The size and shape of the lower jaw retainer piece 202 can be customized to suit the anatomy of each user. The lower jaw retainer piece 202 may include a first fixing member 214 (as shown in FIG. 10) which may be a metallic (e.g. stainless steel) dental wire to ensure that the lower jaw retainer piece 202 securely engages the user's teeth.

The holding member 206 is mounted on the front-facing portion of the lower jaw retainer piece 202 by the first mounting unit 210, and has an opening 222 for receiving a rod 208. The holding member 206 may have a ring shape and be longitudinally perpendicular to a front-facing portion of the lower jaw retainer piece 202. The opening 222 is adapted to receive the rod 208 from the front-facing portion of the lower jaw retainer piece 202 towards the forward direction along the longitudinal axis.

FIG. 11B shows a front portion of an exemplary holding member mounted on a front-facing portion of a lower jaw retainer piece in accordance with at least some embodiments described herein. The holding member 206 may be mounted on or attached to the front-facing portion of the lower jaw retainer piece 202 via a first mounting unit 210. The first mounting unit 210 may be placed into the front-facing portion of the lower jaw retainer piece 202. The rod 208 may be slidably inserted into the opening 222 in FIG. 11B from a backward to a forward direction.

FIG. 12A shows a top portion of an exemplary rod mounted on a front-facing portion of an upper jaw retainer piece in accordance with at least some embodiments described herein. An upper jaw retainer piece 204 is shaped and adapted to engage the maxillary dentition of the user, and the upper jaw retainer piece 204 is open towards a maxillary. The size and shape of the upper jaw retainer piece 204 can be customized to suit the anatomy of each user. The upper jaw retainer piece 204 may include a second fixing member 220 which may be a metallic (e.g. stainless steel) dental wire to ensure that the upper jaw retainer piece 204 securely engages the user's teeth.

One end (i.e. a trailing end 226) of the rod 208 is mounted on the front-facing portion of the upper jaw retainer piece 204 by the second mounting unit 216, and another end (i.e. a leading end 224) is extending along a longitudinal axis in a forward direction. When the rod 208 is assembled with the holding member 206 in accordance with at least some embodiments described herein, the leading end 224 of the rod 208 may be slidably inserted into the opening 222 of the holding member 206 along a longitudinal axis in a forward direction. The oral appliance 200 may optionally include a protective cover 236 that covers at least a part of the rod 208. The protective cover 236 may have a substantially hollow semi-cylindrical shape defining a rounded inner surface. One end of the protective cover 236 is mounted on the front-facing portion of the upper jaw retainer piece 204 via the second mounting unit 216 and above the rod 208 and the other end of the protective cover 236 is extended along a longitudinal axis and parallel to the rod 208. The rounded inner surface of the protective cover 236 faces the rod 208, and a gap exists between the protective cover 236 and the rod 208. FIG. 12B shows a bottom portion of an exemplary rod mounted on a front-facing portion of an upper jaw retainer piece in accordance with at least some embodiments described herein.

In accordance with some implementations, the lower jaw retainer piece 202 and the upper jaw retainer piece 204 may be made of same material as that of the lower jaw retainer piece 102 and the upper jaw retainer piece 104 as mentioned above. Further, the rod 208 and the holding member 206 may be made of aforementioned same material used for forming the rod 108 and the holding member 106.

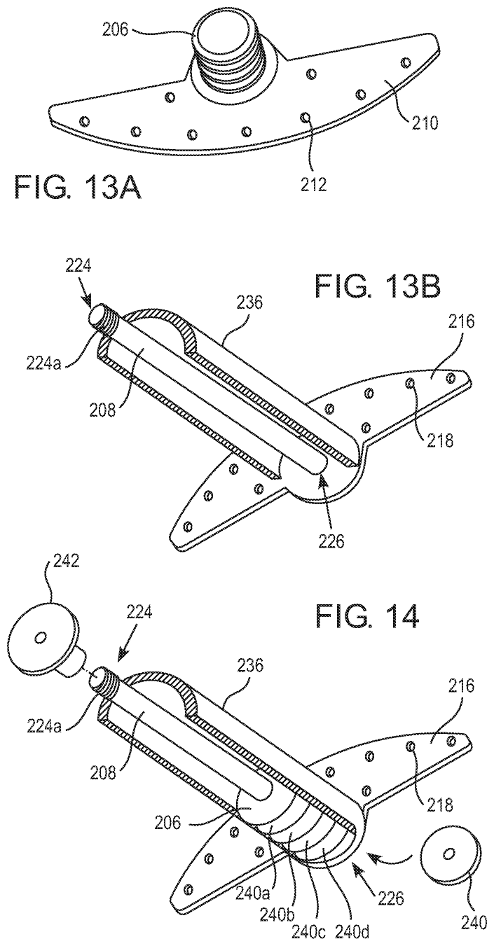

FIG. 13A shows an exemplary holding member 206 and FIG. 13B shows an exemplary rod 208 in accordance with at least some embodiments described herein. One end of the holding member 206 is attached to a first mounting unit 210 that is adapted to be mounted on the front-facing portion of the lower jaw retainer piece 202. The first mounting unit 210 is a thin plate-like member that may be made of same material with the holding member 206. The first mounting unit 210 may be formed in a curved shape along the shape of a front-facing portion of the lower jaw retainer piece 202. The first mounting unit 210 may have a first plurality of through-holes 212 for facilitating the fixation of the first mounting unit 210 to the lower jaw retainer piece 202. The first mounting unit 210 may be mounted on or attached to the front-facing portion of the lower jaw retainer piece 202 using a resin material which may be used for forming the lower jaw retainer piece 202. The resin material may pass through the first plurality of through-holes 212 and attach or fix the first mounting unit 210 to the lower jaw retainer piece 202 and enhance the fixing strength each other. One end of the holding member 206 may be mounted on the front-facing portion of the lower jaw retainer piece 202 via the first mounting unit 210.

The rod 208 is an elongated member that includes a trailing end 226 and a leading end 224. The trailing end 226 of the rod 108 is to be mounted on or attached to the front-facing portion of the upper jaw retainer piece 204, and the leading end 224 is extending along a longitudinal axis in a forward direction. The trailing end 226 of the rod 208 is attached to a second mounting unit 216 that is adapted to be mounted on or attached to the front-facing portion of the upper jaw retainer piece 204. The second mounting unit 216 is a thin plate-like member that may be made of material which may be used for forming the rod 208 or the holding member 206. The second mounting unit 216 may be formed in a curved shape along the shape of a front-facing portion of the upper jaw retainer piece 204. The second mounting unit 216 may have a second plurality of through-holes 218 for facilitating the fixation of the second mounting unit 216 to the upper jaw retainer piece 204. The second mounting unit 216 may be mounted on or attached to the front-facing portion of the upper jaw retainer piece 204 using a resin material which may be used for forming the upper jaw retainer piece 204. The resin material may pass through the second plurality of through-holes 218 and attach or fix the second mounting unit 216 to the upper jaw retainer piece 204 and enhance the fixing strength each other.

The oral appliance 200 may include a protective cover 236 that covers at least a part of the rod 208 as described in the above and shown in FIG. 12A. Further, the leading end 224 of the rod 208 comprises a set of threads 224a which are male threads, and adapted to threadingly engage with another restrictor (as shown in FIG. 14).

FIG. 14 shows an exemplary rod and a restrictor in accordance with at least some embodiments described herein. More particularly, FIG. 14 shows that the rod 208 is inserted into the holding member 206. The leading end 224 of the rod 208 is slidably inserted into the holding member 206, and the rod 208 moves in the forward and backward directions relative to the holding member 206. When the oral appliance 200 includes a protective cover 236 that covers at least a part of the rod 208, the rod 208 is slidably inserted into the holding member 206, and the holding member is located at the gap between the rod 208 and the protective cover 236. Therefore, the protective cover 236 covers and protects both of the holding member 206 and the rod 208. The oral appliance 200 may include a restrictor 240 that restricts sliding of the rod 208 relative within the holding member 206. The restrictor 240 may be a plurality of restrictors, for example, four restrictors 240a-240d as shown in FIG. 14. The restrictor 240 may be a restrictor ring, and the leading end 224 of the rod 208 is slidably inserted into the restrictor 240 (i.e. a restrictor ring). Then, the leading end 224 of the rod 208 is slidably inserted into the holding member 206, and the restrictor 240 may be located between the holding member 206 and the upper jaw retainer piece 204 along a longitudinal axis of the rod 208. By adjusting the number of the restrictor 240, an amount of movement of the holding member 206 relative to the rod 208 in the forward direction can be adjusted. By increasing the number of the restrictor 240 between the holding member 206 and the upper jaw retainer piece 204 along the longitudinal axis of the rod 208, an advancement position of the holding member 206 in a forward direction relative to the rod 208 is getting increased. Thereby, an abutment position of the rod 208 relative to the restrictor 240 can be adjusted.

Further, the oral appliance 200 may include another restrictor 242. The leading end 224 of the rod 208 comprises a set of threads 224a which are male threads, and adapted to threadingly engage with the another restrictor 242. The another restrictor 242 prevents the rod 208 from being fully withdrawn from the holding member 206, and prevents the holding member 206 from being fully withdrawn from the rod 208. The another restrictor 242 may be a restrictor cap having a female threaded portion inside which allows the cap to be removably attached to the leading end 224 of the rod 208 having the set of threads thereon. The another restrictor 242 has a diameter that is larger than a diameter of a ring opening of the holding member 206 so as to prevent the holding member 206 being fully withdrawn from the rod 208.

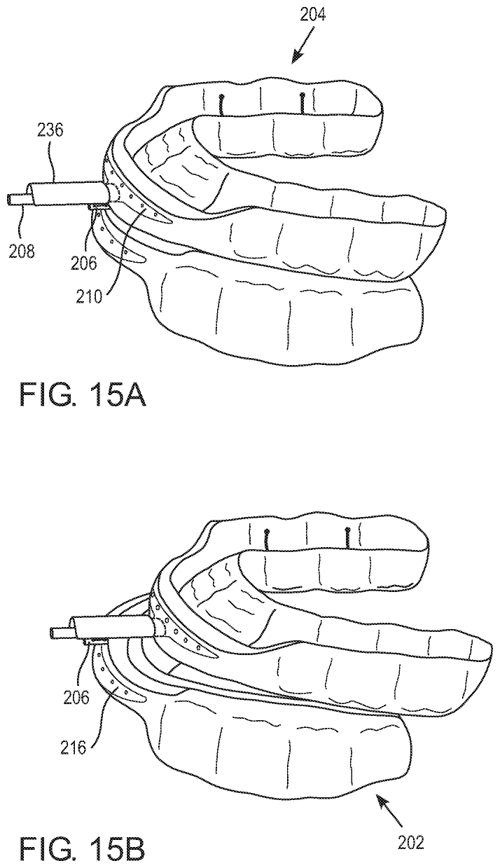

FIGS. 15A and 15B show an exemplary oral appliance while adjusting a lower jaw position in accordance with at least some embodiments described herein. In particular, the assembled oral appliance 200 worn by the user is illustrated in FIGS. 15A and 15B. To assemble the oral appliance 200, as described above, the leading end 224 of the rod 208 may be slidably inserted into the restrictor 240, and then, the leading end 224 may be further slidably inserted into the opening the holding member 206 (a ring shape) from the front-facing portion of the lower jaw retainer piece 202 to a forward direction the along a longitudinal axis. The trailing end 226 of the rod 208 is mounted on the front-facing portion of the upper jaw retainer piece 204. After assembly, the lower jaw position of the user may be adjusted after or before the user wears the oral appliance.

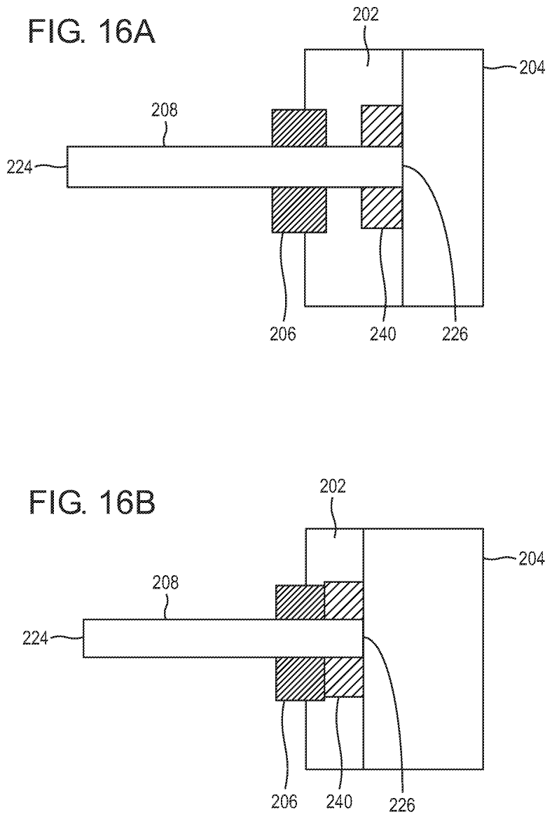

FIG. 15A shows that the position of the lower jaw retainer piece 202 and the position of the upper jaw retainer piece 204 are initially same, and the lower jaw retainer piece 202 has not moved forward yet relative to the upper jaw retainer piece 204. The position of the holding member 206 is adjusted in the forward and backward directions to adjust an abutment position of the rod 208 relative to the restrictor 240 (as shown in FIG. 16A). The position of the holding member 206 relative to the rod 208 is adjusted by the number of the restrictor 240.

As the position of the holding member 206 moves in a forward direction along the rod 208, the position of the lower jaw retainer piece 202 which is attached to the holding member 206 also moves in a forward direction. On the other hand, the position of the upper jaw retainer piece 204 which is attached to the rod 208 maintains same position. Therefore, the lower jaw retainer piece 202 is positioned forward relative to the upper jaw retainer piece 204. FIG. 15B shows that the position of the lower jaw retainer piece 202 and the position of the upper jaw retainer piece 204 are different after sliding and adjustment, and the lower jaw retainer piece 202 has moved in a forward direction relative to the upper jaw retainer piece 204.

By positioning the lower jaw retainer piece 202 forward relative to the upper jaw retainer piece 204, the oral appliance 200 restricts the displacement of the lower jaw of the user to the backward direction, thereby preventing the constriction of the airway, a breathing stop or low respiration during sleep. As the oral appliance 200 adjusts the amount of advancement of the lower jaw retainer piece 202 relative to the upper jaw retainer piece 204, the lower jaw moves forward relative to the upper jaw by the adjusted amount of the advancement. Therefore, it is possible to make the user's airway less likely to be narrowed.

FIG. 16A shows a cross sectional view of an exemplary oral appliance in accordance with at least some embodiments described herein. The oral appliance 200 may include a restrictor 240 that restricts sliding of the rod 208 relative within the holding member 206. For example, the restrictor 240 may be a restrictor ring, and the restrictor 240 is located between the holding member 206 and the upper jaw retainer piece 204 along a longitudinal axis of the rod 208.

An abutment position of the holding member 206 relative to the restrictor 240 is set by sliding the holding member 206 (i.e. a position of the lower jaw retainer piece 202 relative to a position of the upper jaw retainer piece 204). Even after the setting of the abutment position, the holding member 206 can slide forward and backward directions along the rod 208. However, the holding member 206 cannot pass over the restrictor 240 in a backward direction, and the restrictor 240 abuts against the rod 208 and restricts sliding of the holding member 206 in a backward direction relative to the rod 208. Similarly, the rod 208 can move in forward and backward directions relative to the holding member 206 but cannot pass over the holding member 206 in a forward direction because the restrictor 240 abuts against the holding member 206 and restricts a forward movement of the rod 208 relative to the holding member 206. FIG. 16B shows a cross sectional view of an exemplary oral appliance in accordance with at least some embodiments described herein. In particular, a restrictor 240 that abuts against the rod 208 is illustrated in FIG. 16B.

If the oral appliance 200 does not have the restrictor 240, the rod 208 moves in a forward direction, and the holding member 206 moves in a backward direction, and thus, the upper jaw retainer piece 204 attached to the rod 208 can move to a forward direction and the lower jaw retainer piece 202 attached to the holding member 206 can move backward. If the oral appliance 200 does not have the restrictor 240, the lower jaw position of the user who worn the oral appliance 200 during sleeping would not be effectively maintained. The restrictor 240 restricts the displacement of the lower jaw of the user to the backward direction during sleeping and therefore an oral appliance 200 allows the user to maintain the position of the lower jaw forward relative to the upper jaw. At the same time, the restrictor 240 does not restrict the user to move the lower jaw forward relative to the upper jaw when the oral appliance 200 is worn by the user during sleeping. Therefore, the restrictor 240 sets a predetermined degree of a forward and backward movement of mandible within the preferred protruded position, while still maintaining acceptable airway patency effectively by maintaining the mandible in a preferred protruded position. Therefore, the oral appliance 200 allows the user to move the lower jaw forward within a predetermined degree during sleeping, and provides more flexibility, comfortable use, and ease of use of the oral appliance 200 to the user who wears the oral appliance. Thereby, the oral appliance 200 provides great user experience without detriment the effectiveness and function of mandibular advancement.

In accordance with some embodiments, the oral appliance 200 includes another restrictor 242 (as shown in FIG. 14) at opposite side to the restrictor 240. The another restrictor 242 prevents the rod 208 from being fully withdrawn from the holding member 206, and prevents the holding member 206 from being fully withdrawn from the rod 208. The rod 208 has a leading end 224 and a trailing end 226. The leading end 224 of the rod 208 comprises a set of threads 224a which are male threads, and adapted to threadingly engage with the another restrictor 242. The another restrictor 242 may be a restrictor cap having a female threaded portion inside which allows the cap to be removably attached to the leading end 224 of the rod 208 having the set of threads. The another restrictor 242 has a diameter that is larger than a diameter of a ring opening of the holding member 206 so as to prevent the holding member 206 from moving too much in a forward direction and being fully withdrawn from the rod 208. The another restrictor 242 prevents the rod 208 from moving too much in a backward direction and from being fully withdrawn from the holding member 206.

In accordance with some embodiments, the opening 222 of the holding member 208 has an ellipse shape, an oval shape, an egg shape, a rectangle shape, or any oblong shape. Therefore, in the same manner as described above with respect to the opening 122 of the oral appliance 100, the oral appliance 200 further allows the user to move the lower jaw or upper jaw rightward and leftward relative to each other during sleeping. The embodiments described herein improve user experience by allow a predetermined degree of lateral movement of mandible and provide more flexibility, comfortable use, and ease of use of the oral appliance 200 to the user who wears the oral appliance. Accordingly, the oral appliance 200 provides great user experience without detriment the effectiveness and function of mandibular advancement.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

We claim:

1. An oral appliance, comprising:
a first jaw retainer piece;
a second jaw retainer piece;
a holding member mounted on a front-facing portion of the first jaw retainer piece, the holding member having an opening for receiving a rod;
the rod having a leading end slidably inserted into the opening of the holding member and a trailing end mounted on a front-facing portion of the second jaw retainer piece; and
a restrictor that restricts sliding of the rod relative to the holding member.

2. The oral appliance of claim 1, wherein the first jaw retainer piece is a lower jaw retainer piece, and the second jaw retainer piece is an upper jaw retainer piece.

3. The oral appliance of claim 1, wherein the holding member is tubularly shaped and is longitudinally perpendicular to the front-facing portion of the first jaw retainer piece.

4. The oral appliance of claim 1, wherein the holding member has a ring shape.

5. The oral appliance of claim 1, wherein the restrictor abuts against the rod and restricts a forward movement of the rod within the holding member.

6. The oral appliance of claim 3,
wherein the rod comprises a screw portion that extends from the second jaw retainer piece and a screw-adjustable portion that engages the screw portion to move in forward and backward directions, and
wherein when the leading end of the rod is slidably inserted into the holding member, the screw-adjustable portion is adjusted in the forward and backward directions to adjust an abutment position of the rod relative to the restrictor.

7. The oral appliance of claim 1, wherein the restrictor abuts against the rod and restricts a backward movement of the holding member relative to the rod.

8. The oral appliance of claim 3, wherein the restrictor is a protrusion portion formed inside of the holding member.

9. The oral appliance of claim 4, wherein the restrictor is a restrictor ring.

10. The oral appliance of claim 1, further comprising, another restrictor that prevents the rod from being fully withdrawn from the holding member.

11. The oral appliance of claim 1, wherein the opening of the holding member has an ellipse shape, an oval shape, an egg shape, a rectangle shape, or any oblong shape.
